(19) United States
(12) Patent Application Publication
(10) Pub. No.: US 2009/0240143 A1
(43) Pub. Date: Sep. 24, 2009

(54) METHOD AND AN OPTICAL PROBE FOR IN VIVO IMAGING OF A MUCOSA IN A BILIARY OR PANCREATIC SYSTEM AND A METHOD FOR SELECTIVELY OPERATING A TISSUE SAMPLING OF A MUCOSA IN A BILIARY OR PANCREATIC SYSTEM

(75) Inventors: Anne Osdoit, Paris (FR); Magalie Genet, Guyancourt (FR); Nicolas Boularlot, Champigny-sur-Marne (FR); Alexander Meining, Howenbrunn (DE); Christain Prinz, Munchen (DE)

Correspondence Address: OSHA LIANG L.L.P., TWO HOUSTON CENTER, 909 FANNIN, SUITE 3500, HOUSTON, TX, 77010, US

(73) Assignee: Mauna Kea Technologies, Paris (FR)

(21) Appl. No.: 12/396,294

(22) Filed: Mar. 2, 2009

Related U.S. Application Data
(60) Provisional application No. 61/035,795, filed on Mar. 12, 2008.

(51) Int. Cl. A61B 1/06; A61B 6/00; A61B 10/04

(52) U.S. Cl. 600/431; 600/182; 600/160; 600/562

(57) ABSTRACT

A method for observing a mucosa of a biliary or pancreatic system in a subject includes positioning a optical probe in contact with said mucosa, wherein the optical probe accesses the biliary or pancreatic system using a working channel of an endoscope inserted orally in the subject. An optical probe to be used with a fiber optic microscope for in vivo observation includes an optical fiber bundle, a miniaturized objective connected coaxially at a distal tip of the optical fiber bundle, wherein the optical fiber bundle and the miniaturized objective each have a diameter of less than 1.2 mm such that said optical probe can access a biliary or pancreatic system using a working channel of an endoscope inserted orally.

### CROSS-REFERENCE TO RELATED APPLICATIONS

This application claims priority to U.S. Provisional Application Ser. No. 61/035,795, filed on Mar. 12, 2008, which is fully incorporated herein by reference.

### BACKGROUND

1. Field of the Disclosure

The invention relates generally to in vivo imaging of a mucosa in a biliary or a pancreatic system and to tissue sampling of a mucosa in a biliary or a pancreatic system. More specifically, the mucosa may be part of any of a Vater Ampulla, a common bile duct, a common hepatic duct, a right main hepatic duct, a left main hepatic duct, a cystic duct, a pancreatic duct, a gallbladder, a pancreas and a liver.

2. Background Art

Cholangiocarcinoma is a cancer of the bile ducts, which drain bile from the liver into the small intestine. It is a relatively rare cancer, with an annual incidence of 1-2 cases per 100,000 in the Western world. However, the rates of cholangiocarcinoma have been rising worldwide over the past several decades. The symptoms of cholangiocarcinoma include jaundice, weight loss, abdominal pain and sometimes generalized itching. The disease is diagnosed through a combination of blood tests, imaging, endoscopy, and sometimes surgical exploration. Surgery is the only potentially curative treatment, but most patients have advanced and inoperable disease at the time of diagnosis. Prognosis is very poor with a 5-year survival rate of less than 20%.

While abdominal imaging may be useful in the diagnosis of cholangiocarcinoma, direct imaging of the bile ducts is often necessary to differentiate benign from malignant causes of biliary obstruction. Direct imaging of the pancreatic duct is similarly interesting for operating an accurate diagnosis of pancreatic cancer. Current techniques for imaging bile ducts include Endoscopic retrograde cholangiopancreatography (ERCP), an endoscopic procedure performed by a gastroenterologist, which has been widely used for this purpose. Direct cholangiopancreatography may also be accomplished via percutaneous transhepatic insertion of a needle/catheter.

ERCP was introduced for diagnostic evaluation of pancreaticobiliary diseases in late 60's to facilitate radiographic imaging. ERCP is an indirect diagnosis. Basic ERCP procedure consists generally of using an endoscope inserted orally into the duodenum and placing a catheter in the bile/pancreatic duct for injection of a radiographic contrast to provide X-Ray images of the ducts. The goals of ERCP are to detect presence of dilation or narrowing of ducts and to determine cause of such morphologic changes, to obtain fluid or tissue samples and cellular material, and to deliver endoscopic therapeutic interventions, such as sphincterotomy, removal of stones, or placement of stents. Since the introduction of advanced imaging methods, such as MRI and endoscopic ultrasound, ERCP becomes more and more therapeutic. To date, 20% of ERCP procedures are purely diagnostic, while 80% of such procedures are therapeutic. ERCP procedures are now rarely performed without therapeutic intent.

However, accurate diagnosis and staging of biliary and pancreatic cancers remains a clinical problem despite advanced imaging methods currently available. Up to 15% of all suspected cholangiocarcinomas are found to be benign bile duct alterations, the rate of RO-resections is unfavorably low, and methods for tissue sampling show low sensitivity (50%). Radiographic diagnosis of pancreaticobiliary malignancies by ERCP is sensitive but nonspecific. A definitive tissue diagnosis of malignancy may be made during ERCP by using brush cytology, fine needle aspiration, and biopsy, but with a relatively low yield unless several techniques are used at the same time. Therefore, there remains a need for a method and a device for improved diagnosis accuracy of biliary and pancreatic cancers.

### Summary of Claimed Subject Matter

One aspect of the invention relates to methods for observing a mucosa of a biliary or pancreatic system in a subject. A method in accordance with one embodiment of the invention includes positioning a optical probe in contact with said mucosa, wherein the optical probe accesses the biliary or pancreatic system using a working channel of an endoscope inserted orally in the subject.

Another aspect of the invention relates to methods for obtaining a tissue sample from a mucosa of a biliary or pancreatic system in a subject. A method in accordance with one embodiment of the invention includes introducing orally an endoscope down to a major duodenal papilla and aligning the endoscope with the major duodenal papilla;

introducing a tube into a working channel of the endoscope and inserting the tube into the biliary or pancreatic system through the major duodenal papilla; introducing a optical probe into a lumen of said tube, wherein the optical probe is coupled to a microscope; observing in vivo the mucosa using the optical probe and the microscope, wherein said optical probe is positioned in contact with said mucosa; determining an area of interest on said mucosa and stabilizing the tube and the endoscope at a position close to said area; extracting the optical probe out of the lumen of said tube; inserting a tissue sampling tool in the lumen of the tube; and sampling the tissue from said area of interest.

Another aspect of the invention relates to optical probes to be used with a fiber optic microscopes for in vivo observation. An optical probe in accordance with one embodiment of the invention includes an optical fiber bundle, a miniaturized objective connected coaxially at a distal tip of the optical fiber bundle, wherein the optical fiber bundle and the miniaturized objective each have a diameter of less than 1.2 mm such that said optical probe can access a biliary or pancreatic system using a working channel of an endoscope inserted orally.

Other aspects and advantages of the invention will be apparent from the following description and the appended claims.

### BRIEF DESCRIPTION OF DRAWINGS

FIG. 1 illustrates schematically an oral insertion of an endoscope in a duodenum according to an embodiment of the present invention.

FIG. 2 illustrates an introduction of a catheter in a major duodenal papilla according to an embodiment of the present invention.

FIG. 3 illustrates an ERCP procedure according to the prior art.

FIG. 4 are simultaneous radiographic and endoscopic representations resulting from an ERCP procedure according to the prior art.

FIG. 5 are radiographic images of healthy and unhealthy bile ducts resulting from an ERCP procedure according to the prior art.

FIG. 6 illustrates several parts of a distal part of an optical probe according to an embodiment of the present invention.

FIG. 7 illustrates several parts of a distal part of an optical probe according to an embodiment of the present invention.

FIG. 8 illustrates several parts of a distal part of an optical probe according to an embodiment of the present invention.

FIG. 9 illustrates several parts of a distal part of an optical probe according to an embodiment of the present invention.

FIG. 10 illustrates an introduction of a per-oral cholangioscope in a duodenoscope and of an optical probe into the cholangioscope.

FIG. 11 are cholangioscopical images of a patient with a partial biliary obstruction.

FIG. 12 are cholangioscopical images of a patient with a complete biliary obstruction.

FIG. 13 are confocal images of benign strictures of a patient obtained using a confocal fiber microscope according to an embodiment of the present invention.

FIG. 14 are confocal images of malign strictures of a patient obtained using a confocal fiber microscope according to an embodiment of the present invention.

### DETAILED DESCRIPTION

Specific embodiments of the present invention will now be described in detail with reference to the accompanying Figures. Like elements in the various Figures may be denoted by like numerals.

Embodiments of the invention relate to methods and devices capable of obtaining microscopic images of a mucosa in a biliary/pancreatic system. Embodiments of the invention can improve the quality of diagnosis of pancreatic or biliary cancers. Embodiments of the disclosure also relate to a method for tissue sampling, which takes advantage of microscopy to accurately target a tissue to be sampled.

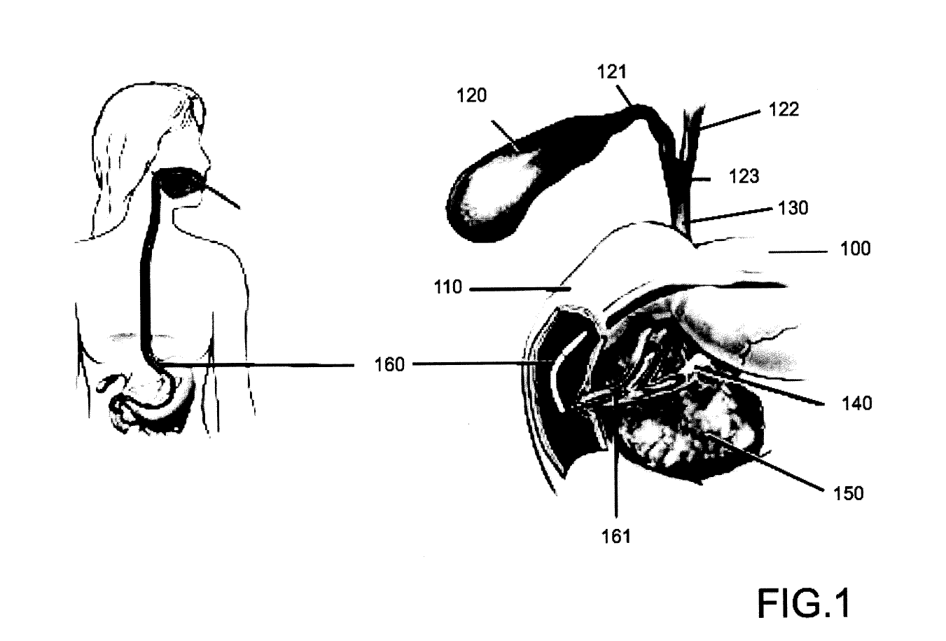

FIG. 1 shows an anatomical illustration of an oral endoscopy with a catheter introduction in a major duodenal papilla of a patient. Such oral endoscopy may be used with embodiments of the present invention. As shown in FIG. 1, an endoscope 160 is orally inserted. The endoscope 160 is gradually advanced down the esophagus and passed through the stomach 100 to reach the duodenum 110. The endoscope 160 may be a duodenoscope, comprising a lateral opening for accessing the major duodenal papilla. FIG. 1 farther illustrates the introduction of a catheter 161 in the papilla at a union between the common bile duct 130 and the pancreatic duct 140. Also shown in FIG. 1 are a gallbladder 120, a cystic duct 121, a common hepatic duct 122, which arises from right and left main hepatic ducts and joins the cystic duct at the level of a junction 123, and a pancreas 150, from which arise the pancreatic duct 140.

In accordance with some embodiments of the present invention, the endoscope 160 may be a duodenoscope. In accordance with other embodiments of the present invention, the endoscope 160 may be a cholangioscope accessing directly the biliary/pancreatic systems. With such a cholangioscope, catheter introduction may not be performed because the cholangioscope may access directly the union between the common bile duct 130 and the pancreatic duct 140. In accordance with such embodiments, a fiber microscope may access the biliary/pancreatic systems through a working channel of a cholangioscope.

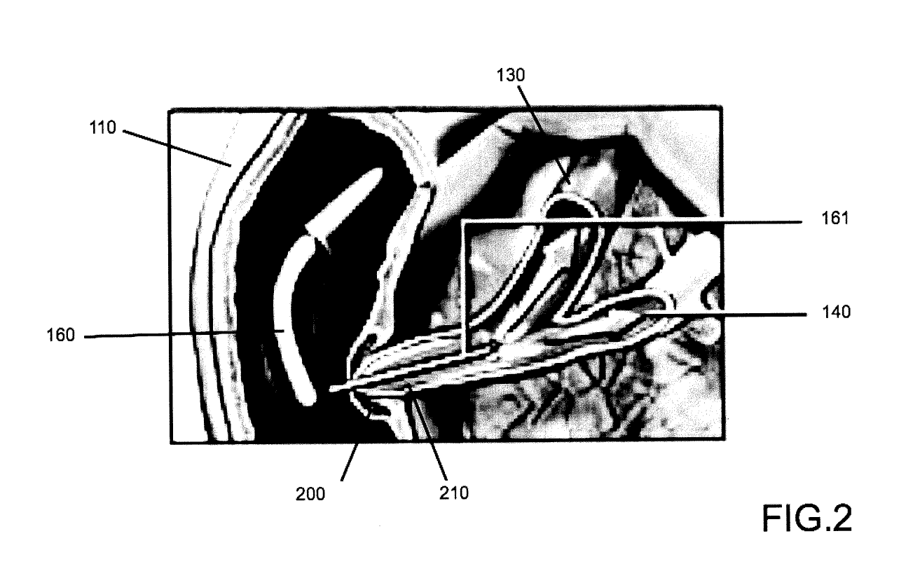

FIG. 2 shows an expanded representation of a catheter introduction, from an endoscope 160 placed in the duodenum 110, into the union of the common bile duct 130 and the pancreatic duct 140. This union is commonly referred to as the Ampulla of Vater 210 and enters the duodenum 110 at the major duodenal papilla 200. In accordance with one embodiment of the present invention, a fiber microscope is inserted in the lumen of the catheter and is then enabled to access a mucosa of the biliary or pancreatic system. The use of a fiber confocal microscope may enable non-superficial microscopic imaging of untreated tissue without previous fixation and preparation of slices. In accordance with another embodiment of the present invention, a per-oral cholangioscope is inserted in a working channel of the endoscope 160 and is further introduced into the union of the common bile duct 130 and the pancreatic duct 140. Per-oral cholangioscope refers to an endoscope accessing the biliary/pancreatic system using a working channel of another endoscope. In accordance with such an embodiment, a fiber microscope may access the biliary/pancreatic system through a working channel of the per-oral cholangioscope.

Introducing a catheter in the major duodenal papilla is also commonly used in the prior art, for example, in an ERCP procedure. Two arrows on FIG. 2 illustrate a possible injection of dye into the pancreatic/bile ducts, which may be operated according to a classic ERCP procedure. The contrast agent may be, for example, an X-ray contrast agent. This injection may be used for further performing X-ray acquisition with a fluoroscope. The introduction of a catheter into the union of the common bile duct and the pancreatic duct is increasingly performed for therapeutic purposes, such as tissue sampling, while ERCP procedures for pure diagnosis purpose are performed at decreasing frequencies.

The biliary system may commonly refer to the Vater Ampulla 210, the common bile duct 130, the right and left main hepatic ducts, the common hepatic duct 122, the cystic duct 121, and the gallbladder 120. The pancreatic system may commonly refer to the pancreatic duct 140 and the pancreas 150.

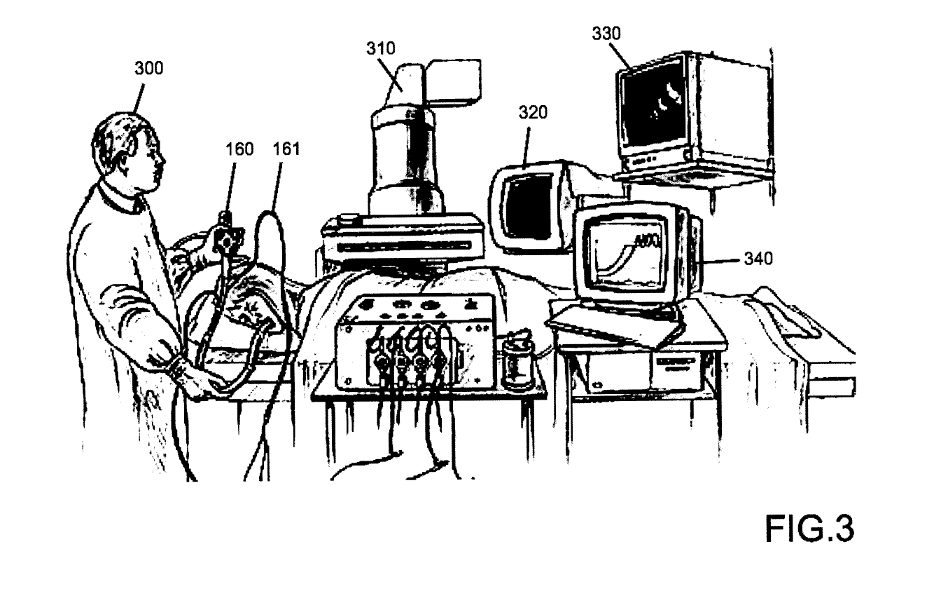

FIG. 3 illustrates several elements coordinated in order to implement an ERCP procedure according to the prior art. The ERCP procedure takes place in a special ERCP suite that has an instant X-ray machine, called a fluoroscope 310. Patient is under conscious heavy sedation. Duration is approximately 30 to 90 minutes. A basic ERCP procedure consists generally of using an endoscope 160 inserted orally into the duodenum and placing a catheter 161 in the bile/pancreatic duct for injection of a radiographic contrast to provide X-Ray images of the ducts. Injection shows indirect effects of bile/pancreatic cancer or stones, such as blockage or dilatation of ducts and inflammation of tissue. An advanced ERCP procedure may further comprise papillotomy and insertion of a special device for cholangioscopy, pancreaticography, stone retrieval, sphincterotomy, drainage, stenting, cytology, or biopsy.

Although ERCP is an invasive procedure with attendant risks, it enables one to obtain biopsies and to place stents or to perform other interventions to relieve biliary/pancreatic obstructions. Endoscopic ultrasound may also be performed at the time of ERCP and may increase the accuracy of the tissue sampling and yield information on lymph node invasion and operability.

Referring still to FIG. 3, a doctor 300 inserts orally a endoscope 160 into a patient. A catheter 161 is introduced into a working channel of the endoscope 160 and steered into the pancreatic/bile ducts using an endoscopic display on an endoscope monitor 330. Injection of an X-ray contrast agent in the bile/pancreatic ducts is carried out and X-ray images from the fluoroscope 310 may be displayed on an X-ray monitor 320. Displays may also be stored on a computer 340.

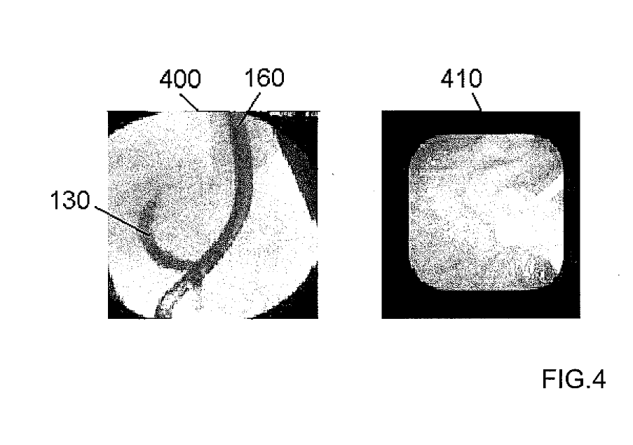

Images 400 and 410 in FIG. 4 are, respectively, radiographic and endoscopic images, which may be obtained through a prior art ERCP procedure, described above with reference to FIG. 3. An endoscope 160 and a common bile duct 130 are visible in image 400.

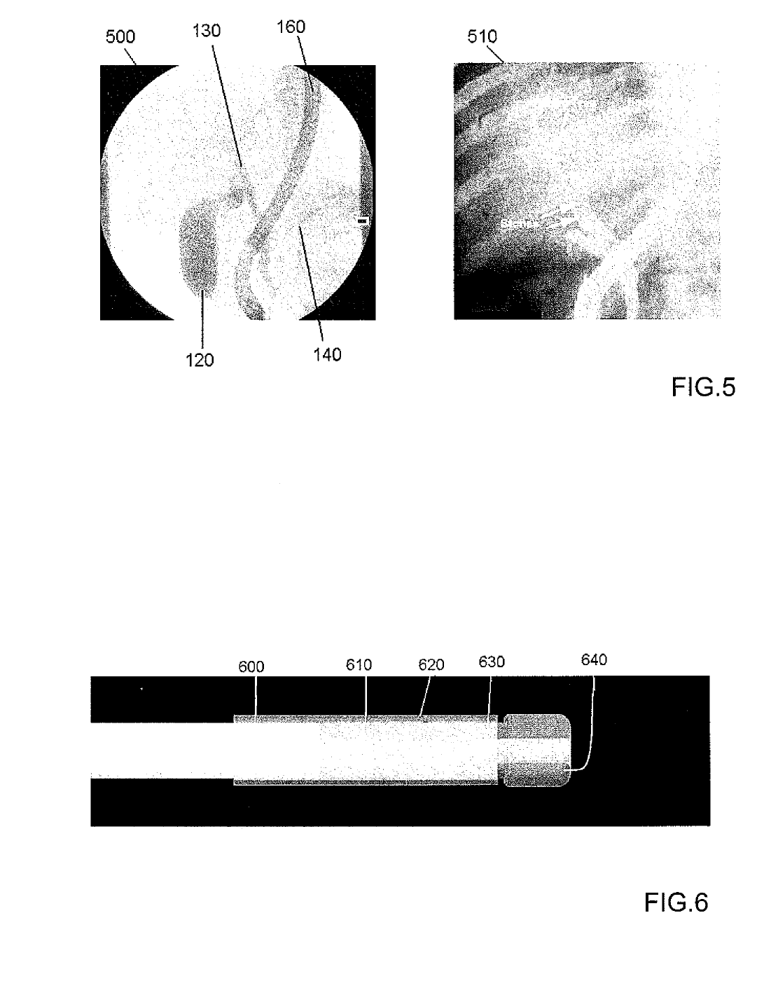

Images 500 and 510 in FIG. 5 are, respectively, radiographic images of healthy and unhealthy bile ducts, which may be obtained through a prior art ERCP procedure, described above with reference to FIG. 3. Biliary ducts and main pancreatic duct 140 observed in image 500 are narrow and a gall bladder 120 is visible without any stones. It is possible to observe stones in the common biliary duct in image 510.

FIG. 6 illustrates several parts of an optical probe distal tip according to an embodiment of the present invention. The optical probe comprises a coherent bundle 610 of several thousands of optical fibers sheathed in a sheath 600. The fibers may, for example, have a core diameter of 2 μm and a mean core spacing of 3.3 μm. A proximal end of the bundle may be connected to a real-time scanning confocal microscope (such as Cellvizio® from Mauna Kea Technologies), and a distal end of the bundle may be equipped with a miniaturized objective. A proximal end may also be connected to any type of fiber microscopes. Confocal capabilities may enable microscopic imaging of upper layers of the epithelium of pancreatic and bile ducts even through bile. Observations at cellular or micro-vascular levels with high sensitivity may also be made possible. The proximal confocal microscope may include an illumination source, which may be a LASER source, capable of exciting endogenous or exogenous fluorophores fluorescence. The proximal confocal microscope may also include a detection channel, which permits collecting and measuring a fluorescence signal. In an embodiment, the confocal microscope is a reflection microscope collecting and measuring backscattered light. The distal objective conjugates the distal end of the fiber bundle with a specific image plane, at a specific working distance when the probe is in contact with a biological tissue.

When illuminated one after another by the proximal scanner, each fiber of the bundle becomes an illumination source of a small volume within the tissue. This illumination may excite endogenous or heterogeneous fluorescence. In addition to functioning as a source of light, the illumination fiber also collects the fluorescence signal and transmit it to the proximal scanner. There, the return beam is spatially filtered and directed to the detection channel. As a result, the probe and its proximal scanner perform a confocal exploration of the tissue.

Referring to FIG. 6, a miniaturized objective 630 at a distal end of the bundle 610 may consist of individual standard lenses, or GRIN lenses, or a combination of both.

This allows adapting the output beam to the application, and thus providing the probe with a resolution, a working distance, a depth of focus that complies with an optical analysis of the tissue and its architecture. A mechanical mount 640 may protect a junction between the optics 630 and the distal tip of the bundle 610, said mechanical mount 640 may also limit the bundle distal end invasiveness. A metallic ferrule 620 may cover the junction between the sheath and the mechanical mount 640. The metallic ferrule 620 may hinder the sheath from shifting or taking off because of constraints applied to the bundle distal end, for example during endoscope insertion or cleaning procedures.

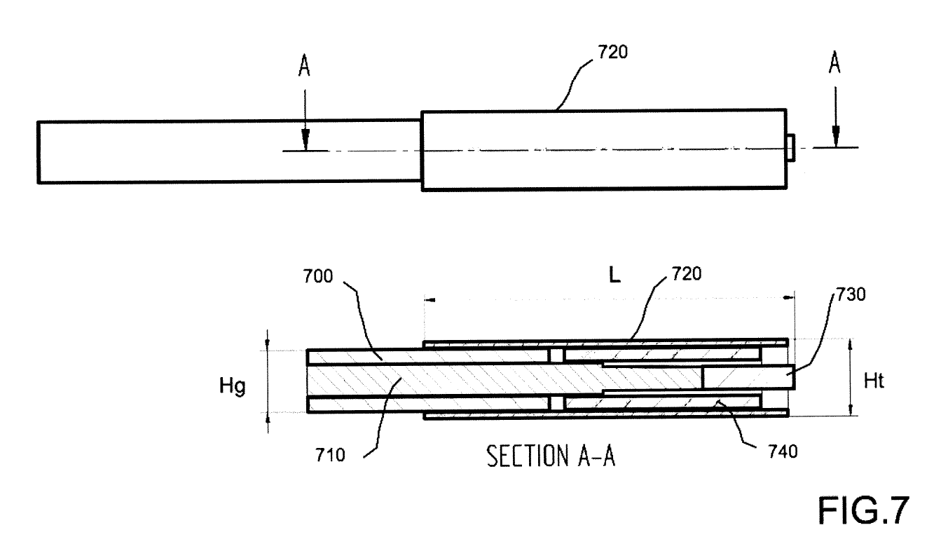

FIG. 7 illustrates a design of an optical probe distal tip according to an embodiment of the present invention. An optical fiber bundle 710 is partially covered (sheathed) in a sheath 700, the distal tip of the optical fiber bundle 710 being not covered by the sheath 700. The external diameter of the sheathed bundle is referred to as Hg in FIGS. 7-9. A miniaturized objective 730 is placed at the distal tip of the optical fiber bundle 710, coaxially with said optical fiber bundle 710. The miniaturized objective 730 may be cylindrical and of a diameter equal to a diameter of the fiber bundle distal tip. A mechanical mount 740 assembles the distal tip of the optical fiber bundle 710 and the miniaturized objective 730, covering the junction of the optical fiber bundle 710 and the miniaturized objective 730. The mechanical mount 740 has a tubular shape, and the distal tip of the optical fiber bundle 710 and the miniaturized objective 730 fit inside the lumen of the mechanical mount 740. The external diameter of the mechanical mount 740 is equal to Hg. A ferrule 720, which may be metallic, assembles the sheath 700 and the mechanical mount 740, covering a portion of the sheath 700 and the mechanical mount 740 over a possible junction. An external diameter of the ferrule 720 is referred to as Ht in FIGS. 7-9. Because of the mechanical mount 740 and/or the ferrule 720, the distal tip of the optical probe thereby presents a rigid part. A length of this rigid part at the optical probe distal tip is referred to as L in FIGS. 7-9.

For accessing the biliary/pancreatic systems, optical fiber probes have to respect severe size requirements as the diameter of common cholangioscopes working channels may be less than 1.2 mm. Miniaturization comes with loss of robustness, resulting from a decrease in contact surfaces between different parts of the distal tip. Despite a very high level of miniaturization, attention may be paid to robustness of the distal tip. Particularly, contact surfaces between the sheath and the ferrule may be kept maximal, in order to provide resistance to traction and to repetitive cleaning and disinfection procedures. For example, the distal end of the fiber bundle may withstand up to twenty manual disinfections and thirty insertions/extractions in endoscopes. In accordance with some embodiments of the invention, the distal end of an optical fiber bundle may also be of single use. In accordance with some embodiments of the invention, the distal end of an optical fiber bundle may be used up to ten times.

On the other hand, in order to keep the steering capabilities of the endoscopes to access bile ducts, and notably because of tip deflections of such endoscopes, optical probe rigid part size may be limited. Common characteristics for optical probes presented in FIGS. 7-9 may be, for example: Depth of observation about 40 to 50 μm, Field of view about 325 μm, lateral resolution about 3.5 μm, axial resolution about 40 μm, rigid part length L from about 4 mm to about 5.6 mm, external diameter Ht from about 0.85 mm to about 1.15 mm. Such miniaturization level enables the tip of the optical probe to be placed in a perpendicular angle to the tissue, or at least tangential rather than in a longitudinal manner in the biliary/pancreatic ducts.

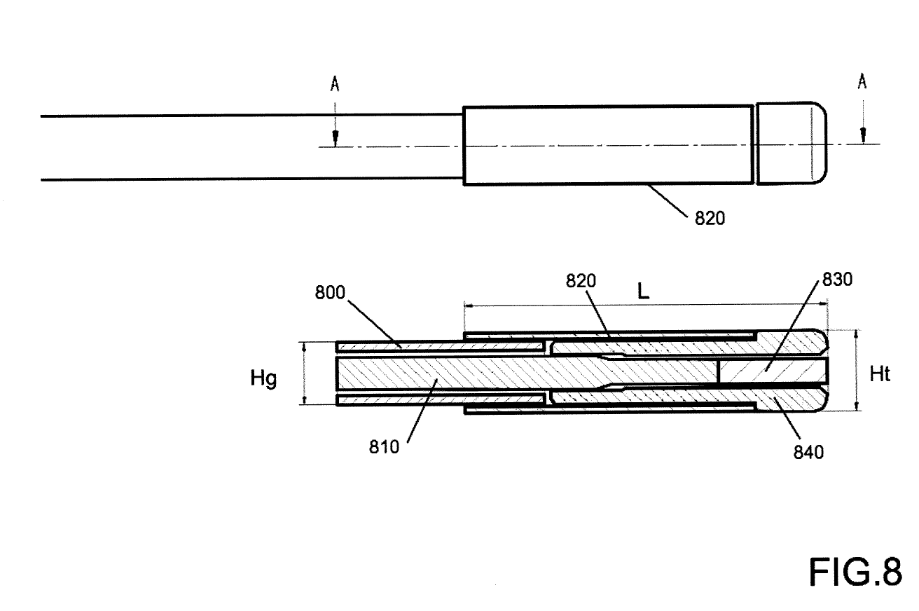

FIG. 8 illustrates a design of an optical probe distal tip according to another embodiment of the present invention. This design comprises five elements: an optical fiber bundle 810, a miniaturized objective 830, a non invasive mechanical mount 840 having a non traumatic form, a ferrule 820 and a sheath 800. The optical fiber bundle 810 is sheathed in the sheath 800, the distal tip of the optical fiber bundle being not covered by said sheath. The miniaturized objective 830 is placed coaxially at the distal tip of the optical fiber bundle 810. The mechanical mount 840 assembles the distal tip of the optical fiber bundle 810 and the miniaturized objective 830, covering a possible junction between these two elements. The mechanical mount 840 extends to the tip of the miniaturized objective 830 that is to be in contact with an observed tissue. At the tip of the miniaturized objective 830, the mechanical mount 840 may have a round (smooth) edge in order to limit its invasiveness, the maximal external size of the mechanical mount being less than or equal to an external diameter of the ferrule 820. The optical fiber bundle 810 and the miniaturized objective 830 may be glued using a glue (e.g., Vitralit ®) using a tool to ensure coaxiality. The mechanical mount 840 may be then glued to the junction between the miniaturized objective 830 and the optical fiber bundle 810 with a glue (e.g., Epotek 301 ®). The ferrule 820, which may be metallic, may be glued on the sheath 800 and the mechanical mount 840 using a glue (e.g., Epotek 301 ®). The length of the rigid part for this design may be, for example, about 5 mm and the external diameter may be of about 0.94 mm.

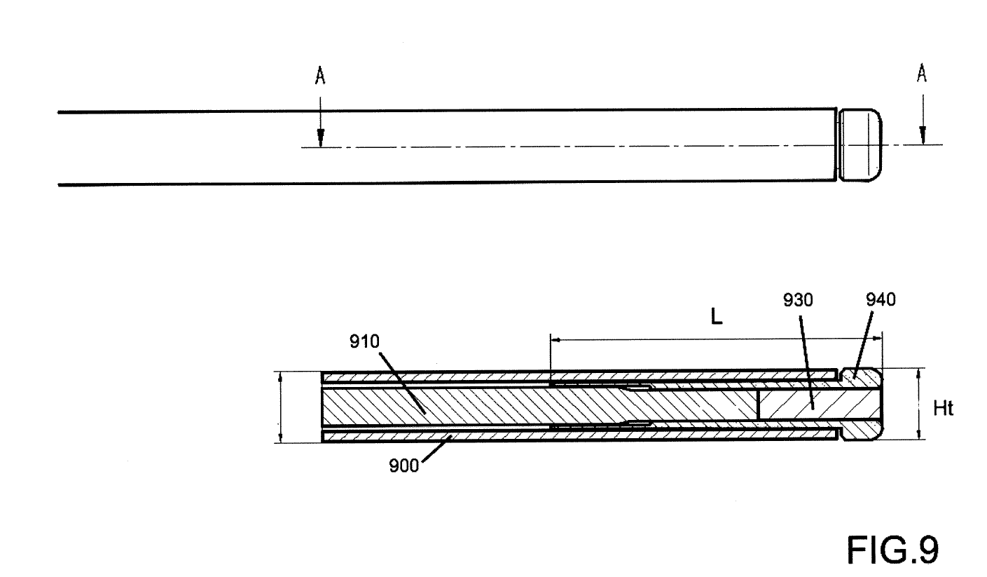

FIG. 9 illustrates a design of an optical probe distal tip according to another embodiment of the present invention. This design comprises four elements: an optical fiber bundle 910, a miniaturized objective 930, a non invasive mechanical mount 940 having a non traumatic form and a sheath 900. The miniaturized objective 930 is placed coaxially at the distal tip of the fiber bundle 910. The mechanical mount 940 assembles the distal tip of the bundle 910 and the miniaturized objective 930, covering a possible junction between these two elements and extending to the tip of the miniaturized objective 930 that is to be in contact with an observed tissue. At the tip of the miniaturized objective 930 to be in contact with the observed tissue, the mechanical mount 940 may have a round (smooth) edge in order to limit its invasiveness. The sheath 900 wrapping the fiber bundle 910 may also cover partially the mechanical mount 940. At the tip of the miniaturized objective 930, the maximal external size of the mechanical mount 940 may be less than or equal to the external diameter of the sheath 900.

A method for manufacturing this design may be to first pull out the sheath in order to strip a bundle distal part, to which is attached a miniaturized objective. The mechanical mount may be then glued to the junction between the bundle distal part and the objective and the sheath may be then pushed to cover the mechanical mount. The bundle and the miniaturized objective may be glued using a glue (e.g., Vitralit ®) using a tool to ensure coaxiality. The mechanical mount may be then glued to the junction between the miniaturized objective and the bundle with a glue (e.g., Epotek 301 ®). Length of the rigid part for this design may be about 4 nm and the external diameter may be of about 0.85 mm.

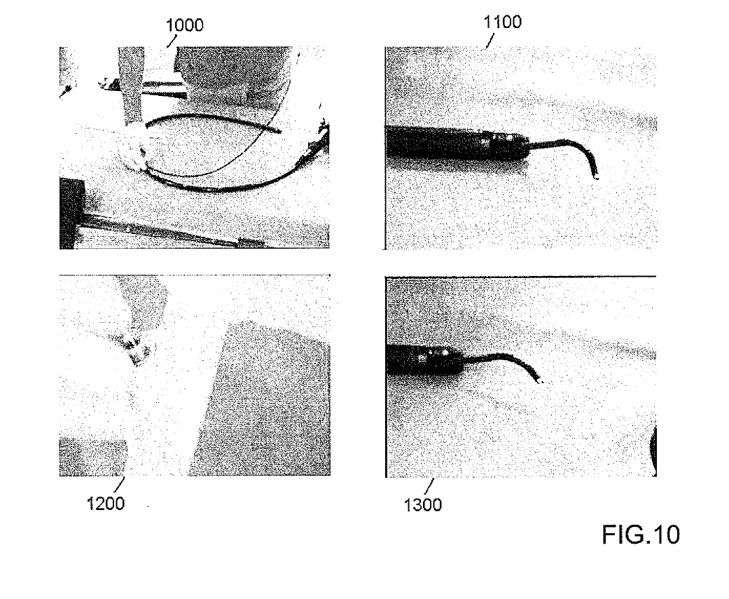

Image 1000 in FIG. 10 illustrates an introduction of a per-oral cholangioscope in a working channel of a duodenoscope. Image 1100 highlights steering capabilities of a deflection tip of the cholangioscope. Image 1200 illustrates an introduction of an optical probe according to embodiments of the present invention in a working channel of a cholangioscope. Image 1300 illustrates steering of the optical probe loaded into the cholangioscope.

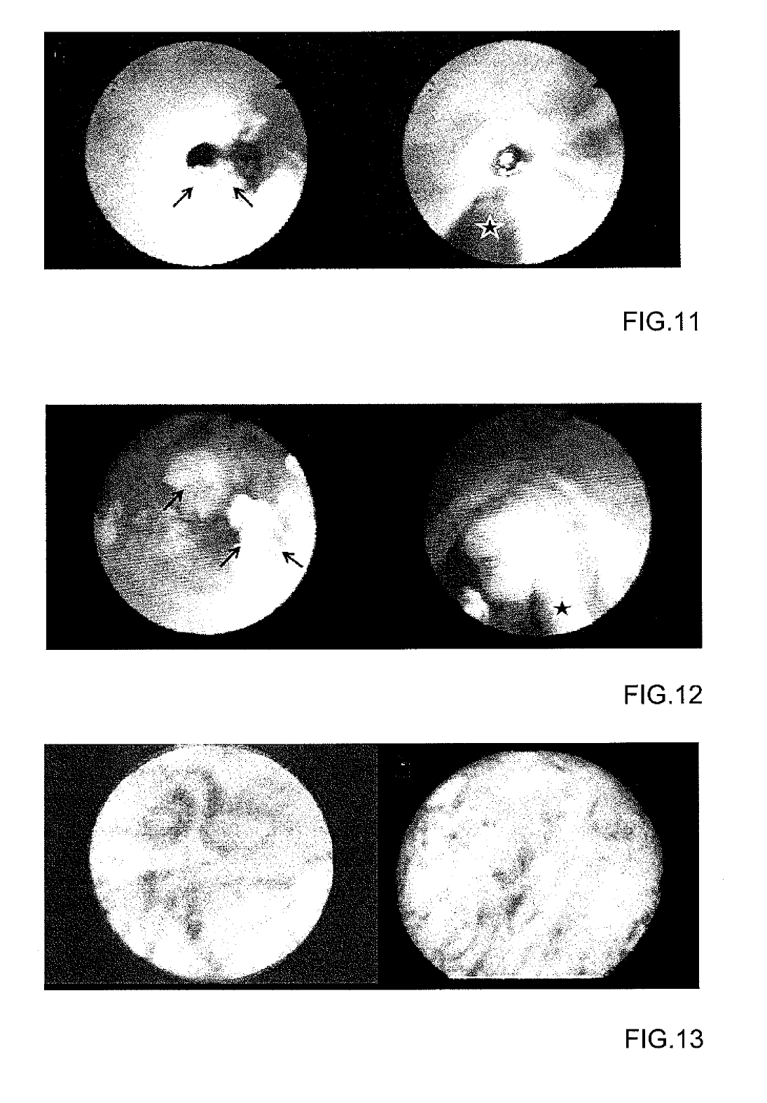

FIG. 11 show cholangioscopical images of a patient with a partial biliary obstruction. The image on the left is an endoscopic image, arrows indicate a small polypoid mass. After endoscopic detection of an area of interest, an optical probe according to embodiments of the present invention may be positioned in said area. On the right image, a star indicates a miniaturized objective according to embodiments of the present invention, positioned on the polypoid mass.

FIG. 12 are cholangioscopical images of a patient with a complete biliary obstruction. The image on the left is an endoscopic image, arrows are indicating a important polypoid tumor obstructing a bile duct. After endoscopic detection of an area of interest, an optical probe according to embodiments of the present invention may be positioned in said area. On the right image, a star indicates the miniaturized objective according to embodiments of the present invention, positioned on the tumor.

FIG. 13 are confocal images of benign strictures of a patient. The images are obtained using a confocal fiber microscope according to an embodiment of the present invention. Benign findings are characterized by reticular arrangement of dark-grey bands on a light-grey background.

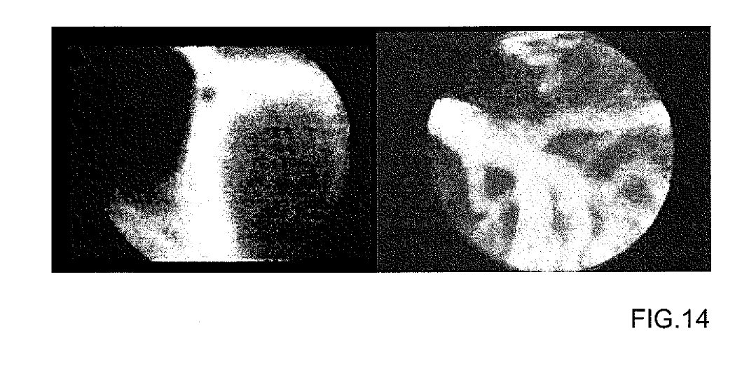

FIG. 14 are confocal images of malignant strictures of a patient. The images are obtained using a confocal fiber microscope according to an embodiment of the present invention. Malign findings are characterized by a black/dark-grey background with irregular large white streaks representing blood vessels filled with fluorescein.

Advantages of embodiments of the invention may include one or more of the following. Embodiments of the invention are compatible with existing fiber-based confocal microscopes, such as Cellvizio® from Mauna Kea Technologies (Paris, France). Miniprobes and methods of the invention can be seamlessly integrated into the workflow of other Glendoscopic procedures. In addition, the new ERCP procedures can also be integrated with existing protocols. The working distances of embodiments of the invention, for example—50 μm—and their confocal capabilities permit microscopic imaging of the upper layers of the epithelium of the pancreatic duct or the bile duct even through the bile. At this depth, observations at cellular or micro-vascular levels with a high sensitivity is made possible. Thanks to their high level of miniaturization, the miniprobes of the invention are fully compatible with the existing procedures, and the miniprobes dimensions are compatibles with cholangioscopes' working channel and/or existing catheter. Miniprobes of the invention are robust; they can withstand at least 20 manual disinfections (standard chemical products) and 30 insertions/extractions in the endoscopes.

While embodiments of the invention has been described with respect to a limited number of embodiments, those skilled in the art, having benefit of this disclosure, will appreciate that other embodiments may be devised which do not depart from the scope of the invention as disclosed herein. Accordingly, the scope of the invention should be limited only by the attached claims.

What is claimed is:

1. A method for observing a mucosa of a biliary or pancreatic system in a subject, comprising:
positioning a optical probe in contact with said mucosa, wherein the optical probe accesses the biliary or pancreatic system using a working channel of an endoscope inserted orally in the subject.

2. The method according to claim 1, wherein the mucosa belongs to one selected from the group consisting of a Vater Ampulla, a common bile duct, a common hepatic duct, a right main hepatic duct, a left main hepatic duct, a cystic duct, a pancreatic duct, a gallbladder, a pancreas and a liver.

3. The method according to claim 1, wherein the endoscope is a cholangioscope accessing directly the biliary or pancreatic system.

4. The method according to claim 1, wherein the optical probe accesses the biliary or pancreatic system comprises:
introducing the endoscope down to a major duodenal papilla and aligning the endoscope with the major duodenal papilla,
introducing a tube into a working channel of the endoscope and inserting the tube into the biliary or pancreatic system through the major duodenal papilla, and
introducing the optical probe into a lumen of said tube.

5. The method according to claim 4, wherein said tube is one of a catheter and a per-oral cholangioscope.

6. The method according to claim 4, wherein positioning the optical probe in contact with the mucosa comprises bringing the tube close to the mucosa.

7. The method according to claim 1, wherein the optical probe is part of a confocal fiber optic microscope.

8. The method according to claim 1, wherein the optical probe is part of a fluorescence confocal fiber optic microscope.

9. The method according to claim 8, further comprising topically administering a fluorescent contrast agent .

10. The method according to claim 8, further comprising intravenously injecting a fluorescent contrast agent.

11. A method for obtaining a tissue sample from a mucosa of a biliary or pancreatic system in a subject, comprising:
introducing orally an endoscope down to a major duodenal papilla and aligning the endoscope with the major duodenal papilla;
introducing a tube into a working channel of the endoscope and inserting the tube into the biliary or pancreatic system through the major duodenal papilla,;
introducing a optical probe into a lumen of said tube, wherein the optical probe is coupled to a microscope;
observing in vivo the mucosa using the optical probe and the microscope, wherein said optical probe is positioned in contact with said mucosa;
determining an area of interest on said mucosa and stabilizing the tube and the endoscope at a position close to said area;
extracting the optical probe out of the lumen of said tube;
inserting a tissue sampling tool in the lumen of the tube; and
sampling the tissue from said area of interest.

12. The method according to claim 11, wherein the optical probe is part of a confocal fiber optic microscope or a fluorescence confocal fiber optic microscope.

13. An optical probe to be used with a fiber optic microscope for in vivo observation comprising:
an optical fiber bundle,
a miniaturized objective connected coaxially at a distal tip of the optical fiber bundle,
wherein the optical fiber bundle and the miniaturized objective each have a diameter of less than 1.2 mm such that said optical probe can access a biliary or pancreatic system using a working channel of an endoscope inserted orally.

14. The optical probe according to claim 13, wherein the optical fiber bundle and the miniaturized objective each have a diameter of less than 1 mm.

15. The optical probe according to claim 13, further comprising a mechanical mount assembling the distal tip of the optical fiber bundle and the miniaturized objective.

16. The optical probe according to claim 15, wherein a length of the mechanical mount is less than 8 mm.

17. The optical probe according to claim 15, further comprising a sheath covering at least one selected from the group consisting of a portion of the optical fiber bundle, a portion of the miniaturized objective, and a portion of the mechanical mount.

18. The optical probe according to claim 15, wherein the mechanical mount extends to a tip of the miniaturized objective to be in contact with an observed tissue and said mechanical mount is profiled to be non-invasive.

19. The optical probe according to claim 15, wherein the optical fiber bundle is sheathed in a sheath, the distal tip of the optical fiber bundle being not covered by the sheath, and further comprising a ferrule assembling the sheath and the mechanical mount.

20. The optical probe of claim 19, wherein a length of a portion of the optical probe distal tip covered with the ferrule or the mechanical mount is less than 8 mm.
